(19) United States
(12) Patent Application Publication
(10) Pub. No.: US 2007/0100199 A1
(43) Pub. Date: May 3, 2007

(54) Apparatus and method of delivering biomaterial to the heart

(76) Inventors: Lilip Lau, Los Altos, CA (US); George Y. Daniloff, Mountain View, CA (US); William G. Mavity, Los Altos, CA (US)

Correspondence Address: FULWIDER PATTON LLP, HOWARD HUGHES CENTER, 6060 CENTER DRIVE, TENTH FLOOR, LOS ANGELES, CA, 90045, US

(21) Appl. No.: 11/208,425

(22) Filed: Nov. 3, 2005

(51) Int. Cl. A61F 2/00

(52) U.S. Cl. 600/37

(57) ABSTRACT

A biomaterial for treating or preventing congestive heart failure is injected into the intrapericardial space of a patient's heart to apply a mild compressive force on the heart. A volume of biomaterial is placed in the intrapericardial space by an injection needle or catheter, adjacent at least the left ventricle, so that the biomaterial applies a compressive force on the myocardium to relieve cardiac wall tension during at least a portion of the cardiac cycle.

### BACKGROUND OF THE INVENTION

The present invention relates to an apparatus and method for treating the heart. More specifically, the invention relates to injection of a biomaterial into the intrapericardial space to treat at least a portion of a patient's heart.

Congestive heart failure (“CHF”) is characterized by the failure of the heart to pump blood at sufficient flow rates to meet the metabolic demand of tissues, especially the demand for oxygen. One characteristic of CHF is remodeling of at least portions of a patient's heart. Remodeling involves physical changes to the size, shape and thickness of the heart wall. For example, a damaged left ventricle may have some localized thinning and stretching of a portion of the myocardium. The thinned portion of the myocardium often is functionally impaired, and other portions of the myocardium attempt to compensate. As a result, the other portions of the myocardium may expand so that the stroke volume of the ventricle is maintained notwithstanding the impaired zone of the myocardium. Such expansion may cause the left ventricle to assume a somewhat spherical shape.

Cardiac remodeling often subjects the heart wall to increased wall tension or stress, which further impairs the heart's functional performance. Often, the heart wall will dilate further in order to compensate for the impairment caused by such increased stress. Thus, a vicious cycle can result, in which dilation leads to further dilation and greater functional impairment.

Historically, congestive heart failure has been managed with a variety of drugs. Devices have also been used to improve cardiac output. For example, left ventricular assist pumps help the heart to pump blood. Multi-chamber pacing has also been employed to optimally synchronize the beating of the heart chambers to improve cardiac output. Various skeletal muscles, such as the latissimus dorsi, have been used to assist ventricular pumping. Researchers and cardiac surgeons have also experimented with prosthetic “girdles” disposed around the heart. One such design is a prosthetic “sock” or “jacket” that is wrapped around the heart.

Although some of the above-discussed devices hold promise, there remains a need in the art for an improved device for treating CHF to prevent a remodeled heart from further remodeling and/or help reverse remodeling of a diseased heart.

Another prior art device includes a cardiac harness configured to fit generally around a patient's heart and to resist expansion of the heart by applying a compressive force thereto. In one such cardiac harness device, multiple rows of spring-like elements or hinges form around the patient's heart and are interconnected by row connectors. The spring-like elements provide a mild compressive force on the epicardial surface of the heart to relieve wall tension. Such cardiac harnesses can be found in any of U.S. Pat. Nos. 6,702,732; 5,595,912; and 6,602,184.

A further prior art device includes providing a cardiac wall tension reduction device configured to fit at least partially around the heart and to exert a generally inwardly directed force on the heart during at least a portion of the cardiac cycle. This prior art device includes a wall tension reduction device formed of a bioabsorbable biomaterial which can be combined with medicaments for restricting growth of scar tissue by the patient's body. The cardiac wall tension reduction device is positioned about the heart so that the tension reduction device reduces the load on the heart until the bioabsorbable biomaterial dissolves in the body. One such device can be found in co-owned and co-pending U.S. Ser. No. 10/338,394 filed Jan. 7, 2003, the entire contents of which are incorporated by reference herein.

Coronary artery disease causes approximately 70% of congestive heart failure. Acute myocardial infarction (“AMI”) due to obstruction of a coronary artery is a common initiating event that can lead ultimately to heart failure. This process by which this occurs is referred to as remodeling and is described in the text Heart Disease, 5^{th} ed., E. Braunwald, Ch. 37 (1997). Remodeling after a myocardial infarction involves two distinct types of physical changes to the size, shape and thickness of the left ventricle. The first, known as infarct expansion, involves a localized thinning and stretching of the myocardium in the infarct zone. This myocardium can go through progressive phases of functional impairment, depending on the severity of the infarction. These phases reflect the underlying myocardial wall motion abnormality and include an initial dyssynchrony, followed by hypokinesis, akinesis, and finally, in cases that result in left ventricular aneurysm, dyskinesis. This dyskinesis has been described as “paradoxical” motion because the infarct zone bulges outward during systole while rest of the left ventricle contracts inward. Consequently, end-systolic volume in dyskinetic hearts increases relative to nondyskinetic hearts.

The second physical characteristic of a remodeling left ventricle is the attempted compensation of noninfarcted region of myocardium for the infarcted region by becoming hyperkinetic and expanding acutely, causing the left ventricle to assume a more spherical shape. This helps to preserve stroke volume after an infarction. These changes increase wall stress in the myocardium of the left ventricle. It is thought that wall tension is one of the most important parameters that stimulate left ventricular remodeling (Pfeffer et al., 1990). In response to increased wall tension or stress, further ventricular dilatation ensues. Thus, a vicious cycle can result, in which dilatation leads to further dilatation and greater functional impairment. A biomaterial as used in the present invention alleviates these problems and promotes reverse remodeling.

The ability of a biomaterial to conform to the heart is also theoretically important in preventing dilated heart failure after acute myocardial infarctions because it may be important to provide reinforcement during systole, especially early systole.

In addition to providing more myocardial sparing over a greater portion of the cardiac cycle, a biomaterial that remains in compressive contact with the heart into systole would counteract the “paradoxical bulging” of the infarct region that occurs in dyskinetic, aneurysmal hearts during systole. This may attenuate infarct expansion and therefore limit the extend of remodeling that further ensues.

### SUMMARY OF THE INVENTION

In accordance with one aspect of the present invention, a method for treating heart failure comprises introducing a substance into the intrapericardial space of the patient's heart in order to reduce cardiac wall tension. In one embodiment, a biomaterial can be introduced into the intrapericardial space to achieve clinically measurable benefits in the treatment of congestive heart failure or other heart condition. The biomaterial can be biocompatible, bioresorbable, non-resorbable, a liquid, a semi-solid, or a solid, and it is configured to have suitable tensile strength and elastic modulus. In the embodiment where the biomaterial is a liquid or semi-solid, the biomaterial is injected into the intrapericardial space by a needle or a catheter inserted into the intrapericardial sac. In the embodiment where the biomaterial is a solid, the solid can be implanted minimally invasively into the intrapericardial space in order to reduce cardiac wall tension. Since the biomaterial is continually subjected to continuous deformation forces by the beating heart, a ratio between the elastic modulus of the myocardium and elastic modulus of the biomaterial is of importance. In one embodiment, the ratio between elastic modulus of the myocardium and the elastic modulus of the biomaterial substance is between twenty to about the actual stiffness of the myocardium itself. In one preferred embodiment, the biomaterial substance provides a compressive force on the myocardium of less than 5 mm Hg and more preferably less than 2 mm Hg. Under certain conditions the biomaterial substance can provide a compressive force on the myocardium of about 1 mm Hg. The amount of compressive force imparted by the biomaterial on the myocardium depends on numerous factors including the composition of the biomaterial and the amount injected. In one embodiment, the volume of biomaterial injected into the intrapercardial space ranges between 1 ml and 1000 ml, and preferably between 50 ml and 200 ml.

As used herein, “intrapericardial space” has a broad meaning and is intended to be used in conjunction with the spirit and scope of the application of a biomaterial to the epicardial surface of the heart or the myocardium. More specifically, under certain conditions, a patient may have had previous open heart surgery in which the pericardium membrane was opened, but not closed so that the intrapericardial space as such may have limited existence. Thus, in referring to the “intrapericardial space” it is intended that the biomaterial be injected or placed in proximity to the myocardium or in contact with the myocardium which may include being in between the pericardium and the epicardial surface of the heart or the myocardium. Further, in those circumstances where the pericardium has not been opened as a result of open heart surgery, there may be very little space between the pericardium and the epicardial surface of the heart or the myocardium. Likewise, it is intended that the “intrapericardial space” under this circumstance include the space between the inside of the pericardium and the epicardial surface of the heart or the myocardium.

In one embodiment of the invention, the biomaterial can be formed in the intrapericardial space in-situ from precursors, monomers or pre-polymers where the precursors, monomers or pre-polymers undergo a phase transition, polymerization or cross-linking. In this embodiment, typically the precursors, monomers or pre-polymers are introduced into the intrapericardial space in a solution or liquid state and they undergo phase transition into a gel, hydrogel, elastomer, semi-solid or solid biomaterial within a period of time, at the delivery site. In one embodiment, the phase transition takes place in a range of about one second up to about twenty minutes at the delivery site. Other phase transition times are possible depending upon various conditions and particular patient needs.

In another embodiment, the biomaterial substance can be pre-formed, gelled, synthesized or otherwise prepared from precursors, monomers or pre-polymers prior to injecting the biomaterial into the intrapericardial space. After the biomaterial is pre-formed, gelled, synthesized or otherwise prepared from precursors, monomers or pre-polymers, the biomaterial is then injected into the intrapericardial space during minimally invasive or open-heart surgery.

Various polymerization and crosslinking reactions can be used to form a biomaterial in the intrapericardial space for the purposes of the invention including radical polymerization, Michael addition polymerization, condensation reactions, and crosslinking reactions between electrophiles and nucleophiles (addition or condensation reactions between electrophiles and nucleophiles).

In one embodiment, the biomaterial can be formed from monomers, oligomers and pre-polymers of synthetic and naturally occurring polymers including hydrophilic and hydrophobic synthetic polymers, small molecular weight crosslinkers having at least two carbon atoms, proteins, polysaccharides, lipids, DNA and their derivatives. Hydrophilic polymers useful in the invention include, but are not limited to: polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propyene glycol and trimethylene glycol substituted with one or more polyalkylene oxides, e.g., mono-, di- and tri-polyoxyethylated glycerol, mono- and di-polyoxyethylated propylene glycol, and mono- and di-polyoxyethylated trimetylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimenthylacrylamide), and poly(N-isopropyl-acrylamide); poly(olefinic alcohol)s such as poly(vinly alcohol); poly(N-vinyl lactams) such as poly(vinyl pyrrolidone), poly(N-vinyl caprolactam), and copolymers thereof; polyoxazonines, including poly(methyloxazoline) and poly(ethyloxazoline); and polyvinylamines.

The hydrophilic polymer component may be a synthetic or naturally occurring hydrophilic polymer. Naturally occurring hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans; e.g., hyaluronic acid; chitin chondroitin sulfate A, B, or C, keratin sulfate, keratosulfate and heparin; and activated polysaccharides such as dextran and starch derivatives.

Hydrophobic polymers, including low molecular weight polyfunctional species, can also be used in the crosslinkable compositions of the invention. Generally, hydrophobic polymers herein contain a relatively small proportion of oxygen and/or nitrogen atoms. Preferred hydrophobic polymers for use in the invention generally have a carbon chain that is no longer than about 14 carbons.

In another embodiment, the polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

In one embodiment, the biomaterials delivered into the intrapericardial space can be formulated to be biodegradable with a half-degradation time ranging from one day to twelve months. The rate of biodegradation can be modulated by incorporating into the biomaterial hydrolysable or biodegradable chemical bonds and biodegradable segments mention in the paragraph above. Typically, hydrogel biomaterials containing up to 30% of water by weight are biodegraded more rapidly than elastomeric biomaterials containing less then 50% of water by weight. A longer lasting elastomeric biomaterials such cellular perfluoroelastomers from CuMedica, Ltd UK or non-degradable biomaterial, Enteryx™, from Boston Scientific are examples of longer lasting biomaterial implants.

In another embodiment, non-resorbable biomaterial compositions are typically comprised of polymers that are substantially insoluble in physiologic liquids. Suitable biocompatible polymers include, by way of example, cellulose acetates (including cellulose diacetate), ethylene vinyl alcohol copolymers, hydrogels (e.g., acrylics), poly(C₁-C₆) acrylates, acrylate copolymers, polyalkyl alkacrylates wherein the alkyl groups independently contain one to six carbon atoms, polyacrylonitrile, polyvinylacetate, cellulose acetate butyrate, nitrocellulose, copolymers of urethane/carbonate, copolymers of styrene/maleic acid, and mixtures thereof. Copolymers of urethane/carbonate include polycarbonates that are diol terminated which are then reacted with a diisocyanate such as methylene bisphenyl diisocyanate to provide for the urethane/carbonate copolymers.

In order to enhance strength of the biomaterial of the invention, it may be generally desirable to add a “tensile strength enhancer” to the adhesive composition. In one embodiment, such tensile strength enhancers preferably comprise micron-size, preferably 5 to 40 microns in diameter and 20 to 5000 microns in length, high tensile strength fibers, usually with glass transition temperatures well above 37° C. Suitable tensile strength enhancers for use in the present invention include, inter alia, collagen fibers, polyglycolide and polylactide fibers, silk fibers as well as other organic tensile strength enhancers and inorganic tensile strength enhancers. A particularly useful tensile strength enhancer is VICRYL™ (polyglycolide:polylactide, 90:10). Suitable tensile strength enhancers are those that have inherent high tensile strength and also can interact by covalent or non-covalent bonds with the polymerized gel network. The tensile strength enhancer should bond to the gel, either mechanically or covalently, in order to provide tensile support.

Further examples of polymers crosslinking reagents, activation chemistries and reaction conditions suitable for forming a biomaterial for the purposes of the invention include those described in U.S. Pat. Nos. 6,312,725; 6,352,710; 6,217,894; 6,818,018; 6,833,408, and U.S. Published Applications 2004/0219214; 2004/0225077; 2004/0009205; 2003/0162841; 2003/0104032; 2004/0002456; and 2002/0114775, all of which are incorporated by reference herein.

In another embodiment, the biomaterial composition of the invention may also be used for localized delivery of various drugs and other biologically active agents in conjunction with the treatment of congestive heart failure or other heart treatment. Drugs, biopharmaceuticals, and any other physiological process modifying agents may be delivered from the biomaterial composition to a local tissue or into systemic circulation after absorption by local tissues. The biomaterial delivered drug, biopharmaceuticals, therapeutic agents or physiological process modifying agents can be anti-infective, anti-inflammatory, anti-proliferative, anti-angiogenic, anti-neoplastic, anti-scaring, scar-inducing, tissue-regenerative, anesthetic, analgesic, immuno-modulating agents and neuro-modulating. Further examples of drugs include those described in U.S. Pat. No. 6,759,431, and U.S. Published Application 2004/0219214 which are incorporated by reference herein.

In one aspect of the invention, the biomaterial delivered into the intrapericardial space comprises a drug for the treatment of congestive heart failure. Examples of drugs used for congestive heart treatment include Furosemide, Hydrochlorothiazide, Metolazone, Digoxin, Dopamine, Dobutamine, Inamrinone, Milrinone, Captopril, Enalapril, Lisinopril, Nitroprusside, Alprostadil, DITPA, or 3, 5-diiodothyropropionic acid.

In yet another aspect of the invention, a biomaterial delivered into the intrapericardial space comprises a fibrosis-inducing agent causing the formation of fibrotic capsule tissue on the surface of the myocardium and in the intrapericardial space. Examples of fibrosis-inducing reagents include chitosan, fibronectin, bleomycin, polylysine, silk protein, talk powder. Other examples of fibrosis inducing agents include those described in U.S. Published Applications 2005/0169958 and 2005/0169959, which are incorporated by reference herein.

In one embodiment of the invention, the biomaterial composition delivered into intrapericardial space is composed of two reactive polymers able to form a hydrogel or elastomer within about one second to about twenty minutes in the intrapericardial space. The two reactive polymers are mixed during delivery of the biomaterial composition in the intrapericardial space using a Y-shaped connector and mixer. Typically, soluble reactive reagents are placed into syringes connected to the Y-connector having a mixing chamber. Upon extrusion of the reactive reagents from syringes, the reagents are mixed with each other as the mixture is delivered onto the target tissue site. An optional gas source (nitrogen, carbon dioxide, compressed air) can be connected to the Y connector to deliver the biomaterial composition in the form of an aerosol. The reaction conditions (such as pH, polymerization initiator concentration, reactive reagent concentration) can be set to allow immediate (less then ten seconds) polymerization or gelation or slow (up to twenty minutes or longer) polymerization or gelation of the biomaterial.

In yet another embodiment, the reactive reagents can be premixed immediately prior to application of the admixture to the tissue site and delivered in the intrapericardial space in the form of a single reagent. The reaction conditions can be set to allow the admixture to remain liquid and extrudable for several minutes, which, typically, is sufficient for completing the delivery of the admixed biomaterial into the intrapericardial space.

In one aspect of the invention, one or both reactive reagents also are reactive with nucleophilic groups on the tissue surface (i.e. amino and sulfhydril groups of proteins on the cell surface and extracellular matrix). Upon delivery, the admixed reactive reagents react with each other and form the biomaterial implant. Some of the electrophilic groups react with nucleophilic groups on the tissue surface forming covalent chemical links between the biomaterial implant and the tissue surface. For example, if such a reactive formulation is delivered into the intrapericardial space and placed in contact with myocardium, the implanted biomaterial can covalently link to the surface of myocardium. Several commercial products in the field of biosurgery, including implantable sealants and adhesives such as CoSeal®, DuraSeal™ and BioGlue™, are based on crosslinking chemistries that also allow the covalent linking of the implant with the tissue surface.

In yet another aspect of the invention, a biomaterial can be formed as a result of the biological or enzymatic crosslinking of biopolymers. For example, many surgical fibrin sealants adhesives are based on the enzymatic crosslinking of blood product derived components. Typically, a preparation containing thrombin and fibrinogen are mixed together at the tissue site. Thrombin converts fibrinogen into fibrin which spontaneously self-assembles into fibrin fibers causing as a result the formation of a hydrogel-like resorbable biomaterial. Among the commercially available products in this category are Tisseel®, VitaGel™, and FloSeal™.

In one embodiment of the invention, the injection of the biomaterial into the intrapericardial space is visualized under fluoroscopy, ECHO, or MRI. To enhance visualization, a contrast agent, typically a biocompatible (non-toxic) radiopaque biomaterial capable of being monitored during injection into a mammalian subject by, for example, radiography, can be added to the biomaterial formulation. The contrast agent can be either water soluble or water insoluble. Examples of water soluble contrast agents include metrizamide, iopamidol, iothalamate sodium, iodomide sodium, and meglumine. Water insoluble contrast agents (i.e., have a water solubility of less than 0.01 milligrams per ml at 20° C.) include tantalum, tantalum oxide and barium sulfate, each of which is commercially available in the proper form for in vivo use and preferably having a particle size of 10 micron or less. Other water insoluble contrast agents include gold, tungsten and platinum powders.

In another embodiment, suitable biocompatible solvents that can be used to deliver non-water soluble polymers include, by way of example, dimethylsulfoxide, analogues/homologues of dimethylsulfoxide, ethanol, ethyl lactate, acetone, and the like. Aqueous mixtures with the biocompatible solvent can also be employed provided that the amount of water employed is sufficiently small that the dissolved polymer precipitates upon injection into a human body. Preferably, the biocompatible solvent is ethyl lactate or dimethylsulfoxide.

In one aspect of the invention, certain compositions are suitable for forming physically (non-covalently) crosslinked gels in the intrapericardial space. Polymers may be crosslinked by either physical or chemical means. Physical crosslinking differs from chemical crosslinking in that the linkages are typically weaker, of lower energy, and often reversible. Thus, physically crosslinked hydrogels often are deformable mechanically. Four fundamental forces have been found to be responsible for producing physical crosslinking: ionic interactions; hydrophobic interactions; hydrogen bonding and Van der Waals forces.

Ionic Interactions Polymers

Physical crosslinking may be intramolecular or intermolecular or in some cases, both. For example, hydrogels can be formed by the ionic interaction of divalent cationic metal ions (such as Ca+2 and Mg+2) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. These crosslinks may be easily reversed by exposure to species that chelate the crosslinking metal ions, for example, ethylene diamine tetraacetic acid. Multifunctional cationic polymers, such as poly(1-lysine), poly(allylamine), poly(ethyleneimine), poly(guanidine), poly(vinyl amine), which contain a plurality of amine functionalities along the backbone, may be used to further induce ionic crosslinks.

Thermoreversible Polymers

Hydrophobic interactions are often able to induce physical entanglement, especially in polymers, that induces increases in viscosity, precipitation, or gelation of polymeric solutions. For example, poly(oxyethylene)-poly(oxypropylene) block copolymers, available under the trade name of PLURONIC.RTM., BASF Corporation, Mount Olive, N.J., are well known to exhibit a thermoreversible behavior in solution. Thus, an aqueous solution of 30% PLURONIC™ F-127 is a relatively low viscosity liquid at 4° C. and forms a pasty gel at physiological temperatures due to hydrophobic interactions. Other block and graft copolymers of water soluble and insoluble polymers exhibit similar effects, for example, copolymers of poly(oxyethylene) with poly(styrene), poly(caprolactone), poly(butadiene) etc.

Recently, Jeong et al. reported biodegradable, in situ gelling poly(ethylene glycol-b-(DL-lactic acid-co-glycolic acid)-b-ethylene glycol), (PEG-PLGA-PEG), triblock copolymers. (See U.S. Pat. No. 6,117,949 incorporated herein by reference) They exhibited promising properties as an injectable drug delivery system. In vivo studies in rats demonstrated that the copolymer gels were still present after one month. During the degradation, the initially transparent gel became opaque due to preferential mass-loss of hydrophilic PEG rich segments. This change in morphology and the generation of an interface or phase might denature the protein drugs or cause cell deterioration in tissue engineering. In vitro release of porcine growth hormone (PGH) and insulin from the in-situ formed gel stopped after releasing 40-50% of loaded proteins.

Techniques to tailor the transition temperature, i.e., the temperature at which an aqueous solution transitions to a gel due to physical linking, are known. For example, the transition temperature may be lowered by increasing the degree of polymerization of the hydrophobic grafted chain or block relative to the hydrophilic block. Increase in the overall polymeric molecular weight, while keeping the hydrophilic lipophilic ratio unchanged also leads to a lower gel transition temperature, because the polymeric chains entangle more effectively. Gels likewise may be obtained at lower relative concentrations compared to polymers with lower molecular weights.

Solutions of other synthetic polymers such as poly(N-alkylacrylamides) also form hydrogels that exhibit thermoreversible behavior and exhibit weak physical crosslinks on warming. During spraying of thermoreversible solutions, cooling of the solutions may be expected from evaporation during atomization. Upon contact with tissue target at physiological temperatures, viscosity is expected to increase from the formation of physical crosslinks.

pH Responsive Polymers

Similarly, pH responsive polymers that have a low viscosity at acidic or basic pH may be employed, and exhibit an increase in viscosity upon reaching neutral pH, for example, due to decreased solubility. Thus, the stability and reactivity of crosslinkable aqueous polymers can be controlled by appropriate selection of pH. For example, degradable esters are stable at pH 3-5, while reactivity for electrophilic-nucleophilic reactions is highest at elevated pHs. Polyanionic polymers such as poly(acrylic acid) or poly(methacrylic acid) possess a low viscosity at acidic pHs that increases as the polymers become more solvated at higher pHs. The solubility and gelation of such polymers further may be controlled by interaction with other water soluble polymers that complex with the polyanionic polymers. For example, it is well known that poly(ethylene oxides) of molecular weight over 2,000 dissolve to form clear solutions in water. When these solutions are mixed with similar clear solutions of poly(methacrylic acid) or poly(acrylic acid), however, thickening, gelation, or precipitation occurs depending on the particular pH and conditions used (for example see Smith et al., “Association reactions for poly(alkylene oxides) and poly(carboxylic acids),” Ind. Eng. Chem., 51:1361 (1959). Thus, a two component aqueous solution system may be selected so that the first component (among other components) consists of poly(acrylic acid) or poly(methacrylic acid) at an elevated pH of around 8-9 and the other component consists of (among other components) a solution of poly(ethylene glycol) at an acidic pH, such that the two solutions on being combined in situ result in an immediate increase in viscosity due to physical crosslinking.

Naturally Occurring Environment-Sensitive Polymers

Physical gelation also may be obtained in several naturally existing polymers. For example, gelatin, which is a hydrolyzed form of collagen, one of the most common physiologically occurring polymers, gels by forming physical crosslinks when cooled from an elevated temperature. Other natural polymers, such as glycosaminoglycans, e.g., hyaluronic acid, contain both anionic and cationic functional groups along each polymeric chain. This allows the formation of both intramolecular as well as intermolecular ionic crosslinks, and is responsible for the thixotropic (or shear thinning) nature of hyaluronic acid. The crosslinks are temporarily disrupted during shear, leading to low apparent viscosities and flow, and reform on the removal of shear, thereby causing the gel to reform.

One embodiment of the invention uses autologous blood clot in combination with other synthetic and longer lasting gel-forming formulations. Collecting 50-100 ml of venous blood from the patient preoperatively may provide material to delivering and clotting it in the pericardial space. The typical clot will resorb in 7 to 14 days. One of potential benefits of autologous clot is that it will contain autologous platelets and growth factors that my have a therapeutic effect on the weakened myocardium.

In one embodiment, the biomaterial changes mechanical properties depending upon the composition of the biomaterial as disclosed above. More specifically, the biomaterial increases in tensile strength after injection into the intrapericardial space thereby transforming from a liquid having very little tensile strength, into a material having greater tensile strength and therefore greater compressive force to relieve cardiac wall stress. One example of such a biomaterial includes a two component aqueous solution consisting of poly(acrylic acid) at an elevated pH of about 8-9 and poly(ethylene glycol) at an acidic pH, such that the two solutions on being combined in situ result in an immediate increase in viscosity due to physical crosslinking. The higher viscosity biomaterial results in a higher tensile strength than before the solutions were combined.

Further features and advantages of the present invention will become apparent to one of skill in the art in view of the Detailed Description of Preferred Embodiments which follows, when considered together with the attached drawings and claims.

### BRIEF DESCRIPTION OF THE DRAWINGS

FIG. 1 is a schematic view of a heart with a prior art cardiac harness placed thereon.

FIGS. 2A-2B illustrate a prior art spring hinge in a relaxed position and under tension.

FIG. 3 is a schematic view of yet another embodiment of a prior art cardiac harness, shown disposed upon a schematically-illustrated heart.

FIG. 4 is a transverse cross-sectional view of the thoracic cavity exposing the heart and the right and left ventricles and including a biomaterial delivery device.

FIG. 5 is a transverse cross-sectional view taken along lines 5-5 of FIG. 4 depicting the delivery catheter having multiple lumens.

FIG. 6 is a transverse cross-sectional view of the thoracic cavity depicting a biomaterial delivery system injecting biomaterial into the intrapericardial space.

FIG. 7 is a schematic of a patient's heart depicting a biomaterial delivery device for injecting biomaterial into the intrapericardial space.

FIG. 8 is a schematic of a patient's heart depicting a biomaterial injected into the intrapericardial space.

FIG. 9 is a schematic of a patient's heart depicting a needle delivery device for injecting biomaterial into the intrapericardial space.

### DETAILED DESCRIPTION OF THE PREFERRED EMBODIMENTS

The present invention relates to a method and apparatus for treating heart failure. As discussed in Applicants' co-pending application entitled “Expandable Cardiac Harness For Treating Congestive Heart Failure,” U.S. Ser. No. 09/634,043, which was filed on Aug. 8, 2000, now U.S. Pat. No. 6,702,732, the entirety of which is hereby expressly incorporated by reference herein, it is anticipated that remodeling of a diseased heart can be resisted or even reversed by alleviating the wall stresses in such a heart. The present invention discloses embodiments and methods reducing cardiac wall tension.

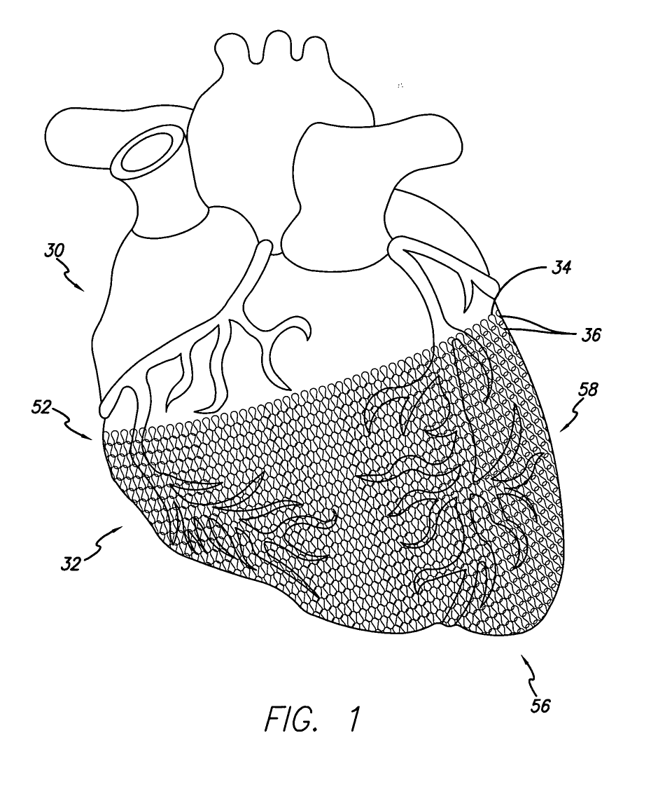

FIG. 1 illustrates a mammalian heart 30 having a prior art cardiac wall stress reduction device in the form of a harness 32 applied to it. The cardiac harness 32 comprises a series of hinges or spring elements 34 that circumscribe the heart 30 and, collectively, apply a mild compressive force on the heart so as to alleviate wall stresses.

The term “cardiac harness” as used herein is a broad term that refers to a device fit onto a patient's heart to apply a compressive force on the heart during at least a portion of the cardiac cycle.

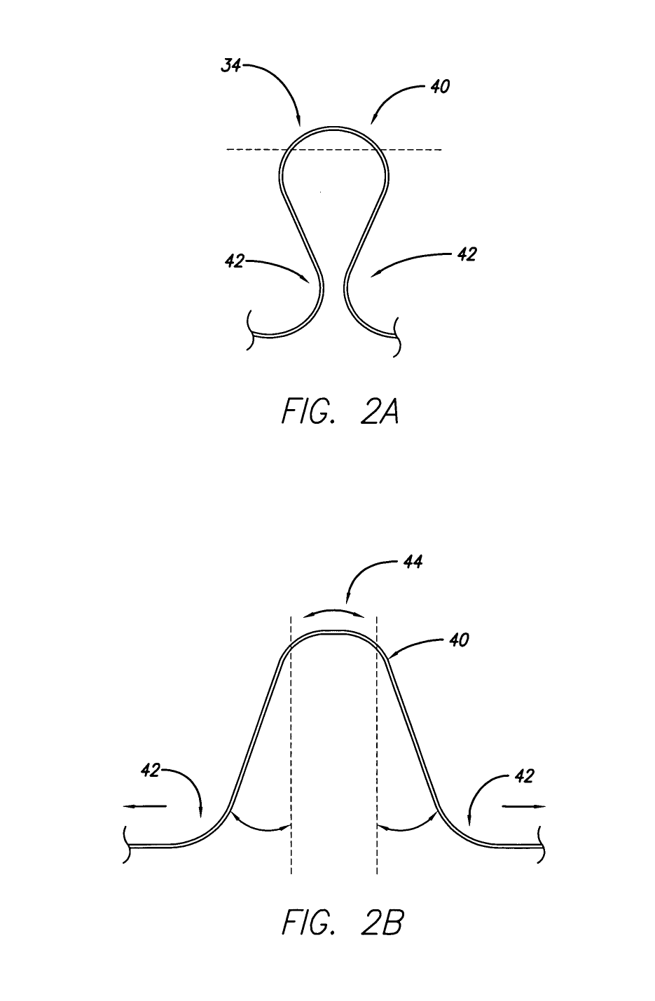

The prior art cardiac harness 32 illustrated in FIG. 1 includes at least one undulating strand 36 comprising a series of spring elements 34 referred to as hinges or spring hinges that are configured to deform as the heart 30 expands during filling. Each hinge 34 provides substantially unidirectional elasticity, in that it acts in one direction and does not provide much elasticity in the direction perpendicular to that direction. For example, FIG. 2A shows one embodiment of a hinge member 34 at rest. The hinge member 34 has a central portion 40 and a pair of arms 42. As the arms are pulled, as shown in FIG. 2B, a bending moment 44 is imposed on the central portion 40. The bending moment 44 urges the hinge member 34 back to its relaxed condition. Note that a typical strand comprises a series of such hinges, and that the hinges 34 are adapted to elastically expand and retract in the direction of the strand 36.

Still referring to FIG. 1, the strands 36 of spring elements 34 are constructed of extruded wire that is deformed to form the spring elements. Although FIG. 1 shows adjacent strands 36 interwoven one with another, it is to be understood that, in additional embodiments, adjacent strands 36 may not overlay or touch one another.

With reference to FIG. 1 the cardiac harnesses 32 has a base portion 52, which is sized and configured to generally engage and fit onto a base region of a patient's heart; an apex portion 56, which is sized and shaped so as to generally engage and fit on an apex region of a patient's heart; and a medial portion 58 between the base and apex portions.

As the heart expands and contracts during diastole and systole, the contractile cells of the myocardium expand and contract. In a diseased heart, the myocardium may expand such that the cells are distressed and lose at least some contractility. Distressed cells are less able to deal with the stresses of expansion and contraction. As such, the effectiveness of heart pumping decreases.

As discussed above, and as discussed in more detail in the applications that are incorporated herein by reference, the undulating spring elements 34 exert a force in resistance to expansion of the heart 30. Collectively, the force exerted by the spring elements tends toward compressing the heart, thus alleviating wall stresses in the heart as the heart expands. Each strand of spring hinges is configured so that as the heart expands during diastole the spring hinges correspondingly expand, storing expansion forces as bending energy in the spring. As such, the stress load on the myocardium is partially relieved by the harness. This reduction in stress helps to decrease the workload of the heart, enabling the heart to more effectively pump blood and helping the myocardium cells to remain healthy and/or regain health.

It is to be understood that several forms of the prior art cardiac harnesses can be constructed that may have varying configurations, sizes, flexibilities, etc., yet still create a mildly compressive force on the heart so as to reduce wall stresses. As discussed in the above-referenced applications, such cardiac harnesses can be constructed from many suitable biomaterials including various metals, fabrics, plastics and braided, woven and/or knit filaments. Suitable biomaterials also include superelastic biomaterials and biomaterials that exhibit shape memory. For example, one such prior art harness is constructed of Nitinol. Shape memory polymers can also be employed. Such shape memory polymers can include shape memory polyurethanes or other polymers such as those containing oligo(e-caprolactone) dimethacrylate and/or poly(e-caprolactone), which are available from mnemoScience. Further, some cardiac harness embodiments substantially encircle the heart, while others may employ spring members disposed over only a portion of the circumference of the heart or harness.

As just discussed, bending stresses are absorbed by the spring members 34 during diastole and are stored in the members as bending energy. During systole, when the heart pumps, the heart muscles contract and the heart becomes smaller. Simultaneously, bending energy stored within the spring members 34 is at least partially released, thereby providing an assist to the heart during systole. The compressive force exerted on the heart by the spring members of the harness comprises about 10% to 15% of the mechanical work done as the heart contracts during systole. Although the harness is not intended to replace ventricular pumping, the harness substantially assists the heart during systole.

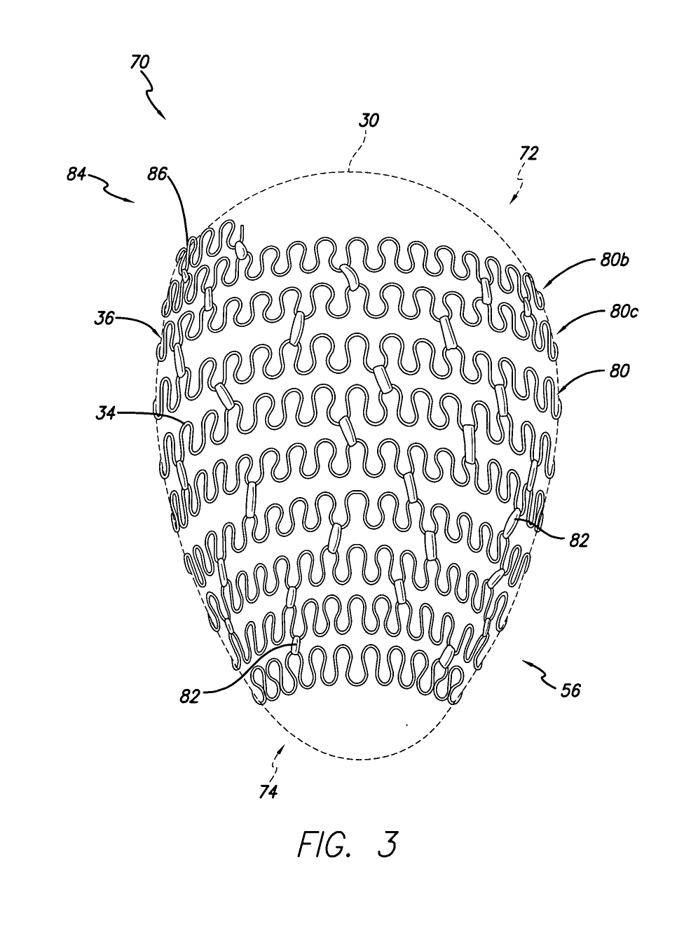

With next reference to FIG. 3, another prior art cardiac harness 70 is illustrated disposed on a schematically illustrated heart 30. As shown; the cardiac harness 70 is configured to circumferentially surround the heart and extend longitudinally from a base portion 72 to an apex portion 74 of the heart. The harness 70 includes a plurality of circumferentially extending rings 80 disposed longitudinally adjacent to one another. Each ring 80 comprises a plurality of interconnected spring members 34. The spring members 34 shown in FIG. 3 are substantially similar to the spring members 34 discussed above with reference to FIGS. 2A and 2B.

A plurality of connectors 82 interconnects adjacent rings 80. The connectors 82 have a length oriented longitudinally relative to the rings so as to create space between adjacent rings. Further, the connectors help to maintain proper alignment between adjacent rings, while allowing some relative movement therebetween. The illustrated harness is configured so that no spring members 34 overlap one another. As such, wear of the harness due to repeated flexing and relative movement of the spring members 34 is avoided. Preferably, the connectors 82 are formed of a semi-compliant biomaterial, such as silicone rubber or other similar biomaterial. It is contemplated that the connectors 82 may include any medical grade polymer such as, but not limited to, polyethylene, polypropylene, polyurethane, nylon, PTFE and ePTFE.

Referring still to FIG. 3, each ring 80 initially comprises an elongate strand having a series of spring members 34. Each strand of spring members 34 preferably is formed of a drawn metallic wire, preferably Nitinol or another metal having a shape memory property. Preferably, the Nitinol wire is shaped into the series of spring members and treated to develop a shape memory of the desired spring member structure. After such treatment, each elongate strand is cut to a length such that when opposite ends of the elongate strand are connected, the elongate strand assumes the ring-shaped configuration shown in FIG. 3.

The prior art cardiac harnesses disclosed herein function very well and provide one form of cardiac wall tension reduction that has proven beneficial to patients in clinical studies. The present invention biomaterial provides a novel and unique form of cardiac wall tension reduction that also should benefit patients having heart ailments including congestive heart failure. The preferred embodiments comprise a biomaterial and apparatus for treating established congestive heart failure as well as for preventing its onset after acute myocardial infarction. Under certain conditions, the biomaterial of the present invention can reverse the remodeling process that occurs in the left and/or right ventricles following a myocardial infarction. Although reference is made throughout the disclosure to treatment of congestive heart failure caused by acute myocardial infarction, the biomaterial of the disclosed embodiments can be used to treat congestive heart failure caused by forward pump failure from any disease such as idiopathic dilated cardiomyopathy, hypertrophic cardiomyopathy, and viral cardiomyopathy. The biomaterial acts by application of a mild compressive force on the left or right ventricle, or both ventricles, to reduce excessive wall tension and to resist shape change of the ventricles during diastole and systole. Use of the biomaterial can attenuate and potentially reverse the remodeling process that occurs in the ventricles following myocardial infarction.

Various polymerization and crosslinking reactions can be used to form a biomaterial in the intrapericardial space for the purposes of the invention including radical polymerization, Michael addition polymerization, condensation reactions, and crosslinking reactions between electrophiles and nucleophiles (addition or condensation reactions between electrophiles and nucleophiles).

In one embodiment, the biomaterial can be formed from monomers, oligomers and pre-polymers of synthetic and naturally occurring polymers including hydrophilic and hydrophobic synthetic polymers, small molecular weight crosslinkers having at least two carbon atoms, proteins, polysaccharides, lipids, DNA and their derivatives. Hydrophilic polymers useful in the invention include, but are not limited to: polyalkylene oxides, particularly polyethylene glycol and poly(ethylene oxide)-poly(propylene oxide) copolymers, including block and random copolymers; polyols such as glycerol, polyglycerol articularly highly branched polyglyerol), propyene glycol and trimethylene glycol substituted with one or more polyalkylene oxides, e.g., mono, di- and tri-polyoxyethylated glycerol, mono- and di-polyoxyethylated propylene glycol, and mono- and di-polyoxyethylated trimetylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimenthylacrylamide), and poly(N-isopropyl-acrylamide); poly(olefinic alcohol)s such as poly(vinly alcohol); poly(N-vinyl lactams) such as poly(vinyl pyrrolidone), poly(N-vinyl caprolactam), and copolymers thereof; polyoxazonines, including poly(methyloxazoline) and poly(ethyloxazoline); and polyvinylamines.

The hydrophilic polymer component may be a synthetic or naturally occurring hydrophilic polymer. Naturally occurring hydrophilic polymers include, but are not limited to: proteins such as collagen, fibronectin, albumins, globulins, fibrinogen, and fibrin, with collagen particularly preferred; carboxylated polysaccharides such as polymannuronic acid and polygalacturonic acid; aminated polysaccharides, particularly the glycosaminoglycans, e.g., hyaluronic acid, chitin, chondroitin sulfate A, B, or C, keratin sulfate, keratosulfate and heparin; and activated polysaccharides such as dextran and starch derivatives.

Hydrophobic polymers, including low molecular weight polyfunctional species, can also be used in the crosslinkable compositions of the invention. Generally, hydrophobic polymers herein contain a relatively small proportion of oxygen and/or nitrogen atoms. Preferred hydrophobic polymers for use in the invention generally have a carbon chain that is no longer than about 14 carbons.

In another embodiment, the polymer may include biodegradable segments and blocks, either distributed throughout the polymer's molecular structure or present as a single block, as in a block copolymer. Biodegradable segments are those that degrade so as to break covalent bonds. Typically, biodegradable segments are segments that are hydrolyzed in the presence of water and/or enzymatically cleaved in situ. Biodegradable segments may be composed of small molecular segments such as ester linkages, anhydride linkages, ortho ester linkages, ortho carbonate linkages, amide linkages, phosphonate linkages, etc. Larger biodegradable “blocks” will generally be composed of oligomeric or polymeric segments incorporated within the hydrophilic polymer. Illustrative oligomeric and polymeric segments that are biodegradable include, by way of example, poly(amino acid) segments, poly(orthoester) segments, poly(orthocarbonate) segments, and the like.

In one embodiment, the biomaterials delivered into the intrapericardial space can be formulated to be biodegradable with a half-degradation time ranging from one day to twelve months. The rate of biodegradation can be modulated by incorporating into the biomaterial hydrolysable or biodegradable chemical bonds and biodegradable segments mention in the paragraph above. Typically, hydrogel biomaterials containing up to 30% of water by weight are biodegraded more rapidly than elastomeric biomaterials containing less then 50% of water by weight. A longer lasting elastomeric biomaterials such cellular perfluoroelastomers from CuMedica, Ltd. UK or non-degradable biomaterial, Enteryx™, from Boston Scientific are examples of longer lasting biomaterial implants.

In another embodiment, non-resorbable biomaterial compositions are typically comprised of polymers that are substantially insoluble in physiologic liquids. Suitable biocompatible polymers include, by way of example, cellulose acetates (including cellulose diacetate), ethylene vinyl alcohol copolymers, hydrogels (e.g., acrylics), poly(C₁-C₆) acrylates, acrylate copolymers, polyalkyl alkacrylates wherein the alkyl groups independently contain one to six carbon atoms, polyacrylonitrile, polyvinylacetate, cellulose acetate butyrate, nitrocellulose, copolymers of urethane/carbonate, copolymers of styrene/maleic acid, and mixtures thereof. Copolymers of urethane/carbonate include polycarbonates that are diol terminated which are then reacted with a diisocyanate such as methylene bisphenyl diisocyanate to provide for the urethane/carbonate copolymers.

In order to enhance strength of the biomaterial of the invention, it may be generally desirable to add a “tensile strength enhancer” to the adhesive composition. In one embodiment, such tensile strength enhancers preferably comprise micron-size, preferably 5 to 40 microns in diameter and 20 to 5000 microns in length, high tensile strength fibers, usually with glass transition temperatures well above 37° C. Suitable tensile strength enhancers for use in the present invention include, inter alia, collagen fibers, polyglycolide and polylactide fibers, silk fibers as well as other organic tensile strength enhancers and inorganic tensile strength enhancers. A particularly useful tensile strength enhancer is VICRYL™ (polyglycolide:polylactide, 90:10). Suitable tensile strength enhancers are those that have inherent high tensile strength and also can interact by covalent or non-covalent bonds with the polymerized gel network. The tensile strength enhancer should bond to the gel, either mechanically or covalently, in order to provide tensile support.

Further examples of polymers, crosslinking reagents, activation chemistries and reaction conditions suitable for forming a biomaterial for the purposes of the invention include those described in U.S. Pat. Nos. 6,312,725; 6,352,710; 6,217,894; 6,818,018; 6,833,408, and U.S. Published Applications 2004/0219214; 2004/0225077; 2004/0009205; 2003/0162841; 2003/0104032; 2004/0002456; and 2002/0114775, all of which are incorporated by reference herein.

In another embodiment, the biomaterial composition of the invention may also be used for localized delivery of various drugs and other biologically active agents in conjunction with the treatment of congestive heart failure or other heart treatment. Drugs, biopharmaceuticals, and any other physiological process modifying agents may be delivered from the biomaterial composition to a local tissue or into systemic circulation after absorption by local tissues. The biomaterial delivered drug, biopharmaceuticals, therapeutic agents or physiological process modifying agents can be anti-infective, anti-inflammatory, anti-proliferative, anti-angiogenic, anti-neoplastic, anti-scaring, scar-inducing, tissue-regenerative, anesthetic, analgesic, immuno-modulating agents and neuro-modulating. Further examples of drugs include those described in U.S. Pat. No. 6,759,431, and U.S. Published Application 2004/0219214 which are incorporated by reference herein.

In one aspect of the invention, the biomaterial delivered into the intrapericardial space comprises a drug for the treatment of congestive heart failure. Examples of drugs used for congestive heart treatment include Furosernide, Hydrochlorothiazide, Metolazone, Digoxin, Dopamine, Dobutarine, Inamrinone, Milrinone, Captopril, Enalapril, Lisinopril, Nitroprusside, Alprostadil, DITPA, or 3, 5-diiodothyropropionic acid.

In yet another aspect of the invention, a biomaterial delivered into the intrapericardial space comprises a fibrosis-inducing agent causing the formation of fibrotic capsule tissue on the surface of the myocardium and in the intrapericardial space. Examples of fibrosis-inducing reagents include chitosan, fibronectin, bleomycin, polylysine, silk protein, talk powder. Other examples of fibrosis-inducing agents include those described in U.S. Published Applications 2005/0169958 and 2005/0169959, which are incorporated by reference herein.

In one embodiment of the invention, the biomaterial composition delivered into intrapericardial space is composed of two reactive polymers able to form a hydrogel or elastomer within about one second to about twenty minutes in the intrapericardial space. The two reactive polymers are mixed during delivery of the biomaterial composition in the intrapericardial space using a Y-shaped connector and mixer. Typically, soluble reactive reagents are placed into syringes connected to the Y-connector having a mixing chamber. Upon extrusion of the reactive reagents from syringes, the reagents are mixed with each other as the mixture is delivered onto the target tissue site. An optional gas source (nitrogen, carbon dioxide, compressed air) can be connected to the Y connector to deliver the biomaterial composition in the form of an aerosol. The reaction conditions (such as pH, polymerization initiator concentration, reactive reagent concentration) can be set to allow immediate (less then ten seconds) polymerization or gelation or slow (up to twenty minutes or longer) polymerization or gelation of the biomaterial.

In yet another embodiment, the reactive reagents can be premixed immediately prior to application of the admixture to the tissue site and delivered in the intrapericardial space in the form of a single reagent. The reaction conditions can be set to allow the admixture to remain liquid and extrudable for several minutes, which, typically, is sufficient for completing the delivery of the admixed biomaterial into the intrapericardial space.

In one aspect of the invention, one or both reactive reagents also are reactive with nucleophilic groups on the tissue surface (i.e. amino and sulfhydril groups of proteins on the cell surface and extracellular matrix). Upon delivery, the admixed reactive reagents react with each other and form the biomaterial implant. Some of the electrophilic groups react with nucleophilic groups on the tissue surface forming covalent chemical links between the biomaterial implant and the tissue surface. For example, if such a reactive formulation is delivered into the intrapericardial space and placed in contact with myocardium, the implanted biomaterial can covalently link to the surface of myocardium. Several commercial products in the field of biosurgery, including implantable sealants and adhesives such as CoSeal® , DuraSeal™ and BioGlue™, are based on crosslinking chemistries that also allow the covalent linking of the implant with the tissue surface.

In yet another aspect of the invention, a biomaterial can be formed as a result of the biological or enzymatic crosslinking of biopolymers. For example, many surgical fibrin sealants adhesives are based on the enzymatic crosslinking of blood product derived components. Typically, a preparation containing thrombin and fibrinogen are mixed together at the tissue site. Thrombin converts fibrinogen into fibrin which spontaneously self-assembles into fibrin fibers causing as a result the formation of a hydrogel-like resorbable biomaterial. Among the commercially available products in this category are Tisseel®, VitaGel™, and FloSeal™.

In one embodiment of the invention, the injection of the biomaterial into the intrapericardial space is visualized under fluoroscopy, ECHO, or MRI. To enhance visualization, a contrast agent, typically a biocompatible (non-toxic) radiopaque biomaterial capable of being monitored during injection into a mammalian subject by, for example, radiography, can be added to the biomaterial formulation. The contrast agent can be either water soluble or water insoluble. Examples of water soluble contrast agents include metrizamide, iopamidol, iothalamate sodium, iodomide sodium, and meglumine. Water insoluble contrast agents (i.e., have a water solubility of less than 0.01 milligrams per ml at 20° C.) include tantalum, tantalum oxide and barium sulfate, each of which is commercially available in the proper form for in vivo use and preferably having a particle size of 10 micron or less. Other water insoluble contrast agents include gold, tungsten and platinum powders.

In another embodiment, suitable biocompatible solvents that can be used to deliver non-water soluble polymers include, by way of example, dimethylsulfoxide, analogues/homologues of dimethylsulfoxide, ethanol, ethyl lactate, acetone, and the like. Aqueous mixtures with the biocompatible solvent can also be employed provided that the amount of water employed is sufficiently small that the dissolved polymer precipitates upon injection into a human body. Preferably, the biocompatible solvent is ethyl lactate or dimethylsulfoxide.

In one aspect of the invention, the biomaterial changes mechanical properties depending on the composition of the biomaterial. For example, certain of the disclosed biomaterial compositions are injected into the intrapericardial space in a liquid form, and after some time period ranging from seconds up to minutes or hours, transform into a more solid material, or a gel-like material having a viscosity higher than a liquid. Under these conditions, the biomaterial changes mechanical properties which may include increased tensile strength after the fluid is injected into the intrapericardial space. The increased tensile strength again depends upon the composition of the biomaterial and will have an effect on the range of compressive forces applied to the myocardium.

In one embodiment of the invention, the injection of the biomaterial into the intrapericardial space is visualized under fluoroscopy, ECHO, or MRI. To enhance visualization, a contrast agent, typically a biocompatible (non-toxic) radiopaque biomaterial capable of being monitored during injection into a mammalian subject by, for example, radiography, can be added to the biomaterial formulation. The contrast agent can be either water soluble or water insoluble. Examples of water soluble contrast agents include metrizamide, iopamidol, iothalamate sodium, iodomide sodium, and meglumine. Water insoluble contrast agents (i.e., have a water solubility of less than 0.01 milligrams per ml at 20° C.) include tantalum, tantalum oxide and barium sulfate, each of which is commercially available in the proper form for in vivo use and preferably having a particle size of 10 micron or less. Other water insoluble contrast agents include gold, tungsten and platinum powders.

In another embodiment, suitable biocompatible solvents that can be used to deliver non-water soluble polymers include, by way of example, dimethylsulfoxide, analogues/homologues of dimethylsulfoxide, ethanol, ethyl lactate, acetone, and the like. Aqueous mixtures with the biocompatible solvent can also be employed provided that the amount of water employed is sufficiently small that the dissolved polymer precipitates upon injection into a human body. Preferably, the biocompatible solvent is ethyl lactate or dimethylsulfoxide.

In one aspect of the invention, certain compositions are suitable for forming physically (non-covalently) crosslinked gels in the intrapericardial space. Polymers may be crosslinked by either physical or chemical means. Physical crosslinking differs from chemical crosslinking in that the linkages are typically weaker, of lower energy, and often reversible. Thus, physically crosslinked hydrogels often are deformable mechanically. Four fundamental forces have been found to be responsible for producing physical crosslinking: ionic interactions; hydrophobic interactions; hydrogen bonding and Van der Waals forces.

Ionic Interactions Polymers

Physical crosslinking may be intramolecular or intermolecular or in some cases, both. For example, hydrogels can be formed by the ionic interaction of divalent cationic metal ions (such as Ca+2 and Mg+2) with ionic polysaccharides such as alginates, xanthan gums, natural gum, agar, agarose, carrageenan, fucoidan, furcellaran, laminaran, hypnea, eucheuma, gum arabic, gum ghatti, gum karaya, gum tragacanth, locust beam gum, arabinogalactan, pectin, and amylopectin. These crosslinks may be easily reversed by exposure to species that chelate the crosslinking metal ions, for example, ethylene diamine tetraacetic acid. Multifunctional cationic polymers, such as poly(1-lysine), poly(allylamine), poly(ethyleneimine), poly(guanidine), poly(vinyl amine), which contain a plurality of amine functionalities along the backbone, may be used to further induce ionic crosslinks.

Thermoreversible Polymers

Hydrophobic interactions are often able to induce physical entanglement, especially in polymers, that induces increases in viscosity, precipitation, or gelation of polymeric solutions. For example, poly(oxyethylene)-poly(oxypropylene) block copolymers, available under the trade name of PLURONIC.RTM., BASF Corporation, Mount Olive, N.J., are well known to exhibit a thermoreversible behavior in solution. Thus, an aqueous solution of 30% PLURONIC™ F-127 is a relatively low viscosity liquid at 4° C. and forms a pasty gel at physiological temperatures due to hydrophobic interactions. Other block and graft copolymers of water soluble and insoluble polymers exhibit similar effects, for example, copolymers of poly(oxyethylene) with poly(styrene), poly(caprolactone), poly(butadiene) etc.

Recently, Jeong et al. reported biodegradable, in situ gelling poly(ethylene glycol-b-(DL-lactic acid-co-glycolic acid)-b-ethylene glycol), (PEG-PLGA-PEG), triblock copolymers. (See U.S. Pat. No. 6,117,949 incorporated herein by reference) They exhibited promising properties as an injectable drug delivery system. In vivo studies in rats demonstrated that the copolymer gels were still present after one month. During the degradation, the initially transparent gel became opaque due to preferential mass-loss of hydrophilic PEG rich segments. This change in morphology and the generation of an interface or phase might denature the protein drugs or cause cell deterioration in tissue engineering. In vitro release of porcine growth hormone (PGH) and insulin from the in-situ formed gel stopped after releasing 40-50% of loaded proteins.

Techniques to tailor the transition temperature, i.e., the temperature at which an aqueous solution transitions to a gel due to physical linking, are known. For example, the transition temperature may be lowered by increasing the degree of polymerization of the hydrophobic grafted chain or block relative to the hydrophilic block. Increase in the overall polymeric molecular weight, while keeping the hydrophilic: lipophilic ratio unchanged also leads to a lower gel transition temperature, because the polymeric chains entangle more effectively. Gels likewise may be obtained at lower relative concentrations compared to polymers with lower molecular weights.

Solutions of other synthetic polymers such as poly(N-alkylacrylamides) also form hydrogels that exhibit thermoreversible behavior and exhibit weak physical crosslinks on warming. During spraying of thermoreversible solution is cooling of the solutions may be expected from evaporation during atomization. Upon contact with tissue target at physiological temperatures, viscosity is expected to increase from the formation of physical crosslinks.

pH Responsive Polymers

Similarly, pH responsive polymers that have a low viscosity at acidic or basic pH may be employed, and exhibit an increase in viscosity upon reaching neutral pH, for example, due to decreased solubility. Thus, the stability and reactivity of crosslinkable aqueous polymers can be controlled by appropriate selection of pH. For example, degradable esters are stable at pH 3-5, while reactivity for electrophilic-nucleophilic reactions is highest at elevated pHs. Polyanionic polymers such as poly(acrylic acid) or poly(methacrylic acid) possess a low viscosity at acidic pHs that increases as the polymers become more solvated at higher pHs. The solubility and gelation of such polymers further may be controlled by interaction with other water soluble polymers that complex with the polyanionic polymers. For example, it is well known that poly(ethylene oxides) of molecular weight over 2,000 dissolve to form clear solutions in water. When these solutions are mixed with similar clear solutions of poly(methacrylic acid) or poly(acrylic acid), however, thickening, gelation, or precipitation occurs depending on the particular pH and conditions used (for example see Smith et al., “Association reactions for poly(alkylene oxides) and poly(carboxylic acids),” Ind. Eng. Chem., 51:1361 (1959). Thus, a two component aqueous solution system may be selected so that the first component (among other components) consists of poly(acrylic acid) or poly(methacrylic acid) at an elevated pH of around 8-9 and the other component consists of (among other components) a solution of poly(ethylene glycol) at an acidic pH, such that the two solutions on being combined in situ result in an immediate increase in viscosity due to physical crosslinking.

Naturally Occurring Environment-Sensitive Polymers

Physical gelation also may be obtained in several naturally existing polymers. For example, gelatin, which is a hydrolyzed form of collagen, one of the most common physiologically occurring polymers, gels by forming physical crosslinks when cooled from an elevated temperature. Other natural polymers, such as glycosaminoglycans, e.g., hyaluronic acid, contain both anionic and cationic functional groups along each polymeric chain. This allows the formation of both intramolecular as well as intermolecular ionic crosslinks, and is responsible for the thixotropic (or shear thinning) nature of hyaluronic acid. The crosslinks are temporarily disrupted during shear, leading to low apparent viscosities and flow, and reform on the removal of shear, thereby causing the gel to reform.

One embodiment of the invention uses autologous blood clot in combination with other synthetic and longer lasting gel-forming formulations. Collecting 50-100 ml of venous blood from the patient preoperatively may provide material to delivering and clotting it in the pericardial space. The typical clot will resorb in 7 to 14 days. One of potential benefits of autologous clot is that it will contain autologous platelets and growth factors that my have a therapeutic effect on the weakened myocardium.

As discussed, when a force is applied to relieve cardiac wall stresses, the working load on the heart is reduced. Reducing the working load allows the heart to at least partially rest, and appears to provide an opportunity for the heart to at least partially heal itself. For example, it is anticipated that a remodeled diseased heart can reverse-remodel so as to become more healthy if cardiac wall stresses are reduced. The effect of reducing wall stress can indeed lead to valuable and beneficial healing effects.

In the embodiments described above, the biomaterial preferably applies a mild compressive force on the heart in order to achieve therapeutic benefits. An applied force or pressure within a therapeutic range is defined herein as a pressure of sufficient magnitude that, when applied to an organ such as the heart, results in a benefit to the organ. In one embodiment, the therapeutic range for the biomaterial is between about 2-20 mmHg. More preferably, the therapeutic pressure is about 2-10 mmHg, and most preferably is between about 2-5 mmHg. Under certain conditions the compressive force of the biomaterial is about 1 mm Hg.

In keeping with the invention, the biomaterial disclosed herein, in any form, can be placed in the intrapericardial space by several methods. It is contemplated that the biomaterial can be injected through the intrapericardial sac by use of a needle that is in fluid communication with the fluid chamber and a plunger. It is also contemplated that a catheter having multiple lumens is delivered into the intrapericardial space where one or more biomaterial fluids are injected into the intrapericardial space to provide the disclosed reduction in cardiac wall tension. Several embodiments are more fully described below.

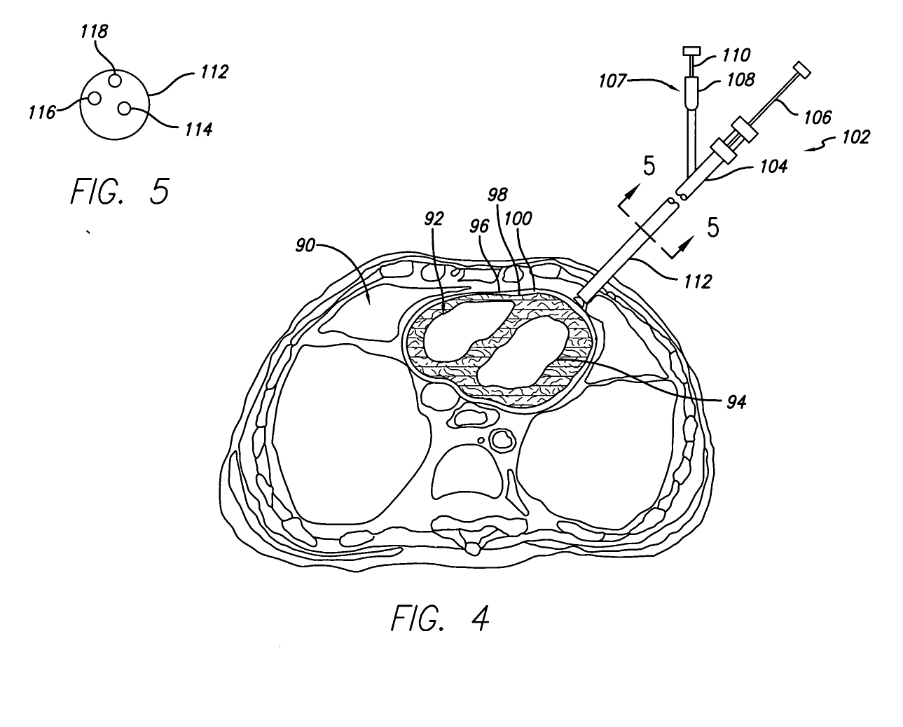

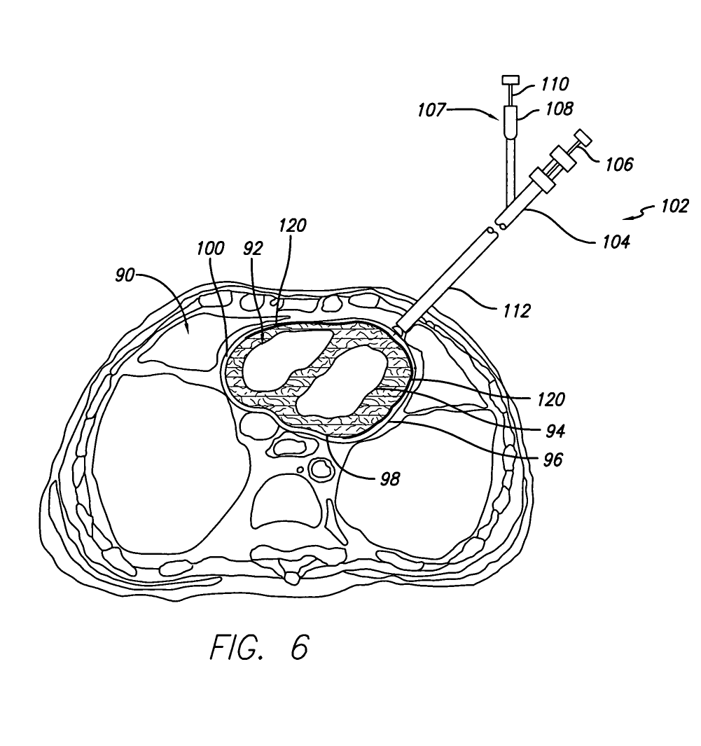

In one embodiment, shown in FIGS. 4-6, the thoracic cavity is shown in transverse cross-section in which the heart 90 is shown with the right ventricle 92 and the left ventricle 94 within the cavity. The pericardium 96 surrounds the heart 90 and the intrapericardial space 100 is the space between the pericardium 96 and the epicardium 98. In this embodiment, a first delivery device 102 can be in the form of a catheter or a needle injection device. A first fluid chamber contains the biomaterial and has a volume that can range between 1 ml up to about 1000 ml. Preferably, the first fluid chamber 104 contains about 50 ml to about 200 ml of biomaterial. A first plunger 106, as shown in FIG. 4, extends proximally away from the first fluid chamber when the first fluid chamber is full of biomaterial. A second delivery device 107 contains a second fluid chamber 108 which can contain a second biomaterial. A second plunger 110 is shown extending proximally away from the second fluid chamber 108 when the second fluid chamber contains biomaterial. The second fluid chamber can contain anywhere from 1 ml up to about 1000 ml of biomaterial, and preferably contains between about 50 ml up to about 200 ml of biomaterial. A catheter body 112 extends from the first delivery device 102 and the second delivery device 107 through the body (between the ribs) and adjacent to or into the pericardium 96. The catheter body 112 has a distal end 113 that either can be inserted through a small incision through the pericardium or can have a sharpened distal end to pierce the pericardium for the purpose of inserting the, distal end 113 of the catheter body through the pericardium 96 and into the intrapericardial space 100. As shown in FIG. 5, the catheter body 112 has multiple lumens, including first lumen 114, second lumen 116, and third lumen 118, which are in fluid communication with first fluid chamber 104 or second fluid chamber 108. Alternatively, the second delivery device 107 could include a vacuum chamber in order to draw a portion of the pericardium into the catheter body 112 so that only a portion of the pericardium is pierced by a needle or other sharp instrument for the purposes of injecting the biomaterial into the intrapericardial space. With reference to FIG. 6, the biomaterial 120 is injected into the intrapericardial space 100 by depressing first plunger 106 and/or second plunger 110. As previously described, the biomaterial 120 may include the combination of several compounds that, when mixed together, results in the formation of a biomaterial that provides the desired cardiac wall stress reduction as previously described. Preferably, the biomaterial 120 is distributed throughout the intrapericardial space 100 and adjacent the left ventricle 94 and/or the right ventricle 92, or both. Further, it is preferred that the biomaterial 120 provide cardiac wall stress reduction during at least a portion of the cardiac cycle, and preferably during both diastole and systole. In one embodiment, a sufficient volume of biomaterial 120 is injected into the intrapericardial space 100 so that the pressure imparted by the biomaterial on the epicardium 98 (or myocardium) is in the range of about 1 mm Hg to about 10 mm Hg. In order to apply the desired compressive force on the epicardium 98, between about 1 ml up to about 1000 ml of biomaterial 120 is injected into the intrapericardial space 100. Preferably, between about 50 ml up to about 200 ml of biomaterial 120 is injected into the intrapericardial space.

Depending upon the form of injection, the pericardium 96 will remain intact and act as a container for the biomaterial 120. If a needle is used to inject the biomaterial 120 there will be no requirement to close any incision in the pencardium 96. In the event a catheter is inserted into the pericardium 96, through a small incision, it may be necessary to close the incision with sutures or staples, which are well known in the art.

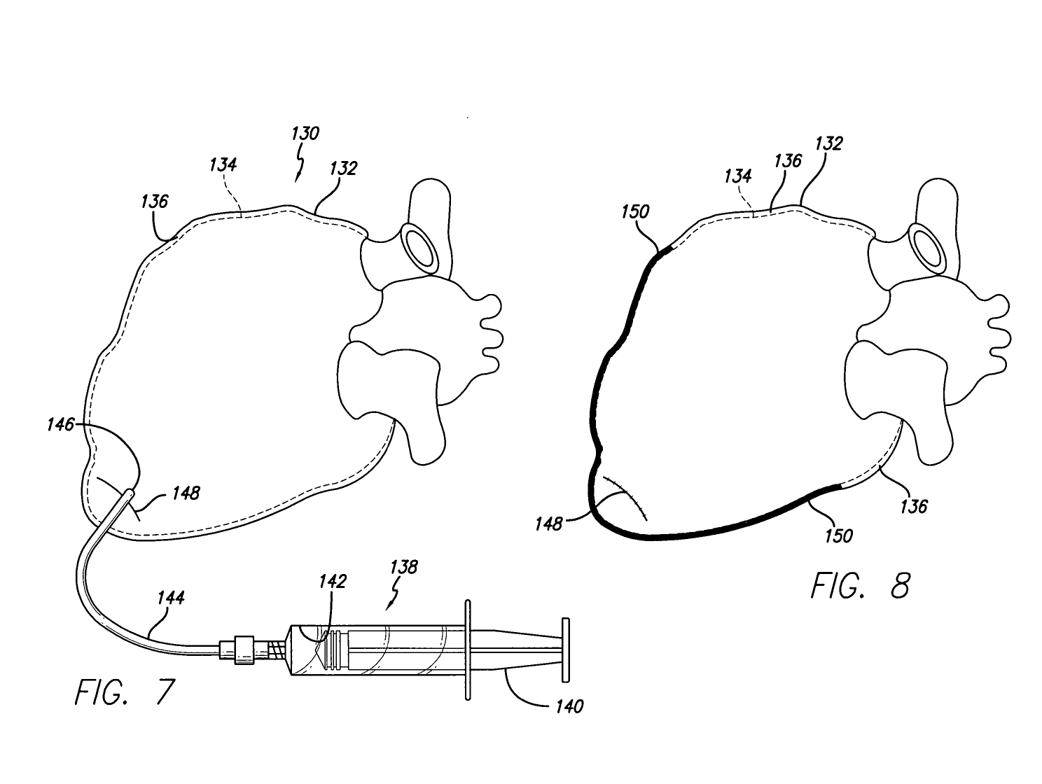

In another embodiment, as shown in FIGS. 7 and 8, the heart 130 is shown with the pericardium 132 covering the epicardium 134. Between the pericardium and the epicardium is the intrapericardial space 136. In this embodiment, catheter delivery system 138 includes a plunger 140 coupled to a fluid chamber 142. The fluid chamber 142 is in fluid communication with catheter 144 that is configured to be inserted into the pericardium 132 through incision 148. The fluid chamber 142 contains a volume of biomaterial 150 as previously described. When the plunger 140 is depressed, it forces the biomaterial 150 out of fluid chamber 142 and through the catheter 144. The catheter distal end 146 extends through the incision and can be placed at various locations within the intrapericardial space 136. More specifically, even though the catheter is intended to be flexible, the catheter 144 has just enough rigidity so that it can be maneuvered throughout the intrapericardial space 136 in order to inject the desired volume of biomaterial 150 at various locations within the intrapericardial space. For example, in one preferred embodiment, the biomaterial 150 is injected in the intrapericardial space 136 adjacent the left ventricle, and in another embodiment the biomaterial 150 is injected in the intrapericardial space adjacent the right ventricle. In another embodiment, the biomaterial is injected within the intrapericardial space 136 so that at least some of the biomaterial is on the epicardial surface 134 and adjacent both the right and left ventricles. The volume of biomaterial 150 injected into the intrapericardial space 136 can range from about 1 ml up to about 1000 ml, and preferably between about 50 ml up to about 200 ml. Depending upon the composition of the biomaterial 150 and the volume of biomaterial injected, the compressive force of the biomaterial on the myocardium is about 1 mm Hg to about 10 mm Hg. Preferably, the biomaterial 150 provides a compressive force on the myocardium during at least a portion of the cardiac cycle, and preferably during diastole. Further, more preferably the biomaterial 150 provides a compressive force on the myocardium during both diastole and systole. As shown in FIG. 8, the incision 148 has been closed by suturing or stapling, which is well known in the art. The biomaterial 150 is distributed throughout the intrapericardial space 136. The catheter delivery system 138 has been removed from the patient's body.

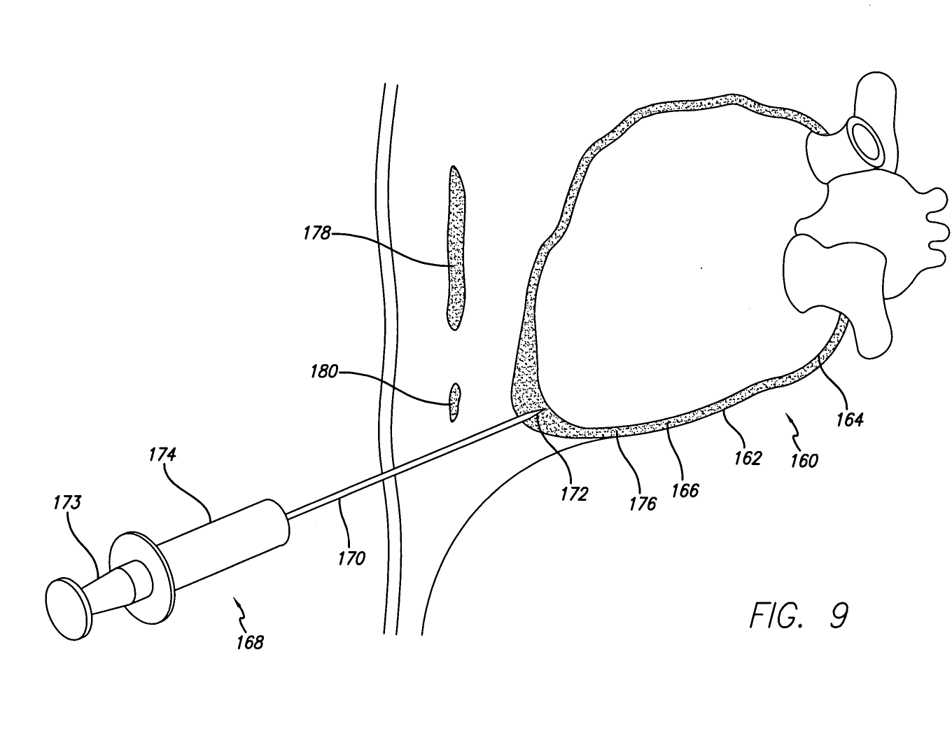

In another embodiment, as shown in FIG. 9, the heart 160 is shown with the pericardium 162 covering the epicardium 164. Between the pericardium and the epicardium is the intrapericardial space 166. In this embodiment, a needle delivery system 168 includes a metallic needle 170 having a sharp pointed end 172 and a fluid chamber 174. A plunger 173 is used to fill and empty the fluid chamber 174. The fluid chamber 174 is in fluid communication with the needle 170 and the sharpened pointed end 172. The fluid chamber 174 contains a volume of biomaterial 176 as previously described. When the plunger 173 is depressed, it forces the biomaterial 176 out of fluid chamber 174 and through the needle 170, and out of the sharpened pointed end 172. The pointed end 172 extends through the skin in a subxyphoid approach just below the sternum 178 and the xyphoid 180. Once the pointed end 172 of the needle 170 pierces the pericardium 162, care must be taken so that the sharpened pointed end does not penetrate the epicardium 164. The needle can be advanced to any location in the intrapericardial space 166 in order to deliver the biomaterial 176 and the position of the pointed end 172 can be monitored under fluoroscopy or by other visualization means. In the subxyphoid approach, the skin is shaved and prepared in a septic fashion and both the skin and subcutaneous tissue can be anesthetized. The skin can be pierced with any surgical blade below and to the left of the xyphoid process, and the subcutaneous tissues can be separated with a clamp. The needle 170 is inserted through the incision and advanced until the pointed end 172 is in the pericardium 162. The needle can have multiple lumens (not shown), or be equipped with a three-way stopcock (not shown) and hooked to an anesthesia reservoir in order to inject anesthetic as the needle is advanced into the pericardium 162. After the pointed end 172 enters the pericardium 162, the biomaterial is injected into the intrapericardial space 166 adjacent either the left ventricle, the right ventricle, or adjacent both ventricles. The volume of biomaterial 176 injected into the intrapericardial space 166 can range from about 1 ml up to about 1000 ml, and preferably between about 50 ml up to about 200 ml. Depending upon the composition of the biomaterial 176 and the volume of the biomaterial injected, the compressive force of the biomaterial on the myocardium is about 1 mm Hg to about 10 mm Hg. Preferably, the biomaterial 150 provides a compressive force on the myocardium during at least a portion of the cardiac cycle, and preferably during diastole. Further, more preferably, the biomaterial 176 provides a compressive force on the myocardium during both diastole and systole. After the biomaterial 176 has been injected into the intrapericardial space 166, the needle 170 is withdrawn from the patient and any incision is closed by conventional means such as sutures or staples. Since the needle 170 is intended to be a small gauge needle, it should be unnecessary to close the injection site in the pericardium 162. If the injection site in the pericardium 162 does require closing, any conventional suture or staple means can be used.

While the delivery systems disclosed herein are intended to be inserted minimally invasively or percutaneously, the delivery systems can be used during open heart surgery when the patient's chest is opened via a median sternotomy. Preferably, however, delivery of the biomaterial by the delivery systems disclosed herein is through minimally invasive surgical access to the thoracic cavity. Access to the thoracic cavity can be between the patient's ribs to gain direct access to the heart. Preferably, such a minimally invasive procedure is accomplished on a beating heart without the use of cardio-pulmonary bypass. Other access sites to the heart are available including subxyphoid access and percutaneous access.

The biomaterial disclosed herein can be used in conjunction with the cardiac harness previously disclosed herein. For example, it is contemplated that the cardiac harness is mounted on the patient's heart as previously described, and the biomaterial is then injected over the cardiac harness to provide an added benefit of reducing cardiac wall stress reduction.

Although this invention has been disclosed in the context of several preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments may be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed:

1. A biocompatible material for treating the heart, comprising:
a biomaterial being placed in the intrapericardial space of the heart; and
a volume of the biomaterial being placed in the intrapericardial space in an amount sufficient to create a compressive force on the myocardium during at least a portion of the cardiac cycle.

2. The biocompatible material of claim 1, wherein the volume of biomaterial placed in the intrapericardial space creates the compressive force on the myocardium in the range of about 1 mm Hg to about 10 mm Hg.

3. The biocompatible material of claim 2, wherein the volume of biomaterial is placed in the intrapericardial space is in the range of about 1 ml to about 1000 ml.

4. The biocompatible material of claim 2, wherein the volume of biomaterial placed in the intrapericardial space is in the range of about 50 ml to about 200 ml.

5. The biocompatible material of claim 2, wherein the volume of biomaterial is configured in the intrapericardial space to provide the compressive force during diastole and systole.

6. The biocompatible material of claim 5, wherein the biomaterial is configured to cover at least the epicardium adjacent to at least the left ventricle.

7. The biocompatible material of claim 5, wherein the biomaterial is configured to cover the epicardium adjacent to at least the right ventricle.

8. The biocompatible material of claim 1, wherein the biomaterial is a solution of a high molecular weight polymer.

9. The biocompatible material of claim 1, wherein the biomaterial is a hydrogel.

10. The biocompatible material of claim 1, wherein the biomaterial is an elastomer.

11. The biocompatible material of claim 1, wherein the biomaterial is comprised of a synthetic polymer.

12. The biocompatible material of claim 1, wherein the biomaterial is comprised of a naturally occurring polymer.

13. The biocompatible material of claim 1, wherein the biomaterial is comprised of a drug or biological process altering agent.

14. The biocompatible material of claim 1, wherein the biomaterial is comprised of a drug used for the treatment of congestive heart condition.

15. The biocompatible material of claim 1, wherein the biomaterial is comprised of a fibrosis-inducing agent.

16. The biocompatible material of claim 1, wherein the biomaterial is bioresorbable.

17. The biocompatible material of claim 1, wherein the biomaterial is bioresorbable within 30 days.

18. The biocompatible material of claim 1, wherein the biomaterial is non-bioresorbable.

19. The biocompatible material of claim 1, wherein the biomaterial is further comprised of a radiopaque contrast reagent.

20. The biocompatible material of claim 1, wherein the material is further comprised of an opaque contrast reagent visible by ultrasound.

21. The biocompatible material of claim 1, wherein the biomaterial is further comprised of a magnetic resonance imaging label.

22. The biocompatible material of claim 1, wherein the biomaterial is placed in the intrapericardial space as two reactive reagents.

23. The biocompatible material of claim 1, wherein the biomaterial is able to react with a tissue surface thereby forming a covalent bond.

24. The biocompatible material of claim 1, wherein the biomaterial is a shear-thinning fluid.

25. The biocompatible material of claim 1, wherein the biomaterial is a thermo-reversible fluid.

26. The biocompatible material of claim 1, wherein the biomaterial is a fibrin sealant.

27. An assembly for treating the heart, comprising:
an elongated injection device having a fluid chamber and having a distal region for insertion into the intrapericardial space of the heart; and
a biomaterial contained in the fluid chamber for injection into the intrapericardial space.

28. The assembly of claim 27, wherein the injection device is an elongated catheter.

29. The assembly of claim 28, wherein the catheter has multiple lumens extending from the fluid chamber to the distal region for injecting the biomaterial into the intrapericardial space.

30. The assembly of claim 27, wherein the fluid chamber has a volume in the range of about 1 ml to about 1000 ml.

31. The assembly of claim 27, wherein the fluid chamber has a volume in the range of about 50 ml to about 200 ml.

32. A method for treating the heart, comprising:
providing a biomaterial;
placing the biomaterial in an intrapericardial space; and
wherein the biomaterial exerts a compressive force on the myocardium during at least a portion of the cardiac cycle.

33. The method of claim 32, wherein the biomaterial exerts the compressive force on the myocardium in the range of about 1 mm Hg to about 10 mm Hg.

34. The method of claim 32, wherein the biomaterial is injected into the intrapericardial space.

35. The method of claim 32, wherein a volume of the biomaterial is placed in the intrapericardial space, the volume ranging from about 1 ml to about 1000 ml.

36. The method of claim 32, wherein a volume of the biomaterial is placed in the intrapericardial space, the volume ranging from about 50 ml to about 200 ml.

37. The method of claim 32, wherein the biomaterial is placed on the myocardium adjacent at least the left ventricle.

38. The method of claim 32, wherein the biomaterial is placed on the myocardium adjacent at least the right ventricle.

39. The method of claim 32, wherein the biomaterial is taken from the group of biomaterials consisting of CoSeal@, DuraSeal™, BioGlue™, Tisseel®, VitaGel™, FloSeal™, and Enteryx™.

40. The method of claim 32, wherein the biomaterial is perfluoroelastomer.

41. The method of claim 32, wherein the biomaterial is silicon.

42. The method of claim 32, wherein placing the biomaterial in the intrapericardial space comprises piercing the pericardium at multiple locations for injecting the biomaterial.

43. The method for treating the heart, comprising:
providing a biomaterial;
placing the biomaterial adjacent the myocardium of the heart; and
wherein the biomaterial exerts a compressive force on the myocardium during at least a portion of the cardiac cycle.

44. The method of claim 43, wherein the biomaterial exerts the compressive force on the myocardium in the range 1 mm Hg to about 10 m Hg.

45. The method of claim 43, wherein the biomaterial is injected adjacent the myocardium.

46. The method of claim 43, wherein a volume of the biomaterial is placed adjacent the myocardium, the volume ranging from about 1 ml to about 1000 ml.

47. The method of claim 43, wherein a volume of the biomaterial is placed adjacent the myocardium, the volume ranging from about 50 ml up to about 200 ml.
